(19) United States
(12) Patent Application Publication
(10) Pub. No.: US 2025/0134647 A1
(43) Pub. Date: May 1, 2025

(54) ENHANCED MONOFOCAL LIGHT ADJUSTABLE INTRAOCULAR LENS

(71) Applicant: RxSight, Inc., Aliso Viejo, CA (US)

(72) Inventors: John Kondis, Irvine, CA (US); Ilya Goldshleger, Ladera Ranch, CA (US)

(21) Appl. No.: 18/498,086

(22) Filed: Oct. 31, 2023

(51) Int. Cl. A61F 2/16

(52) U.S. Cl. CPC A61F 2/1613

(57) ABSTRACT

In some embodiments of an enhanced monofocal intraocular lens (EMF IOL), an optical power of the EMF IOL is characterized by a base optical power for distance vision, consistent with a monofocal lens; and an additive add-power structure for near vision, including a central add-power ring around an optical axis of the IOL; and an axial power hole at the optical axis of the IOL. In embodiments, the add-power structure is induced by an approximate super Gaussian optical path difference, the super Gaussian having a radial coordinate raised to a power greater than two in its exponential. In some EMF IOLs, an optical path difference of the EMF IOL is characterized by a base wavefront for distance vision; and an additive add-power structure wavefront for near vision, approximately characterized by a super Gaussian, having a radial coordinate r raised to a power greater than two in its exponential.

### TECHNICAL FIELD

This application is directed to monofocal intraocular lenses, in more detail to enhanced monofocal intraocular lenses that are light adjustable.

### BACKGROUND

Exchanging the cataract-clouded natural lens of a eye with an intraocular lens (IOL) is a life changing experience for the patients, as these IOLs restore the visual acuity of the patients to the quality they enjoyed early in their lives. However, these IOLs are non-accommodating, and thus providing good vision for the patients both at distance and at near remains a challenge. Various solutions have been proposed to provide good vision at different distances, such as multifocal IOLs and extended depth-of-focus IOLs. Some of these ideas are implemented with diffractive optical designs, others with zonal, or refractive designs. Still, no single optical design emerged as clearly superior to all others and thus there is a persistent need to develop additional intraocular lenses that further improve the patients' visual acuity at different distances.

### SUMMARY

In some embodiments of an enhanced monofocal (EMF) intraocular lens (IOL), an optical power of the EMF IOL is characterized by a base optical power for distance vision, consistent with a monofocal lens; and an additive add-power structure for near vision, including a central add-power ring around an optical axis of the IOL; and an axial power hole at the optical axis of the IOL. In embodiments, the add-power structure is induced by an approximate super Gaussian optical path difference, the super Gaussian having a radial coordinate raised to a power greater than two in its exponential.

In some embodiments of an enhanced monofocal (EMF) intraocular lens (IOL) an optical path difference W(r) of the EMF IOL is characterized by a base wavefront W_{b}(r) for distance vision; and an additive add-power structure wavefront Wₐ(r) for near vision, approximately characterized by a super Gaussian W_{sG}(r), having a radial coordinate r raised to a power greater than two in its exponential.

### BRIEF DESCRIPTION OF THE DRAWINGS

FIGS. 1A-H show beams, log MAR and MTF characteristics of existing IOLs.

FIGS. 2A-B show embodiments of the Enhanced Monofocal (EMF) IOLs 100.

FIG. 3 shows a super Gaussian embodiment of the EMF IOL 100 with zero added paraxial add-power.

FIG. 4 shows a modified super Gaussian embodiment of the EMF IOL 100 with a non-zero added paraxial add-power.

FIG. 5 illustrates the root mean square difference between two wavefronts.

FIG. 6 shows a comparison graph of the defocus-dependent log MAR of various IOLs.

FIG. 7 shows a comparison graph of the defocus-dependent MTF of various IOLs.

### DETAILED DESCRIPTION

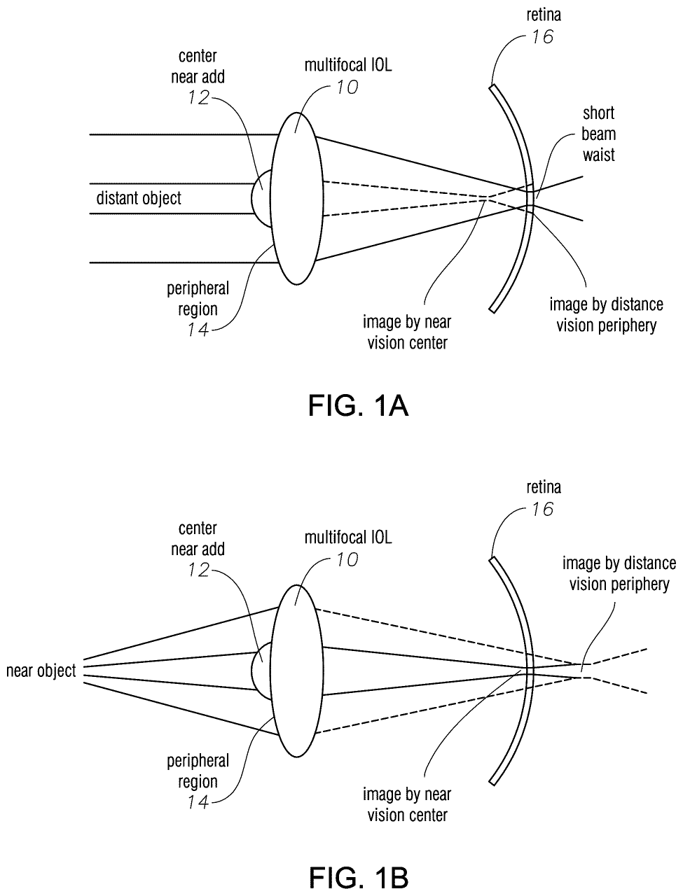

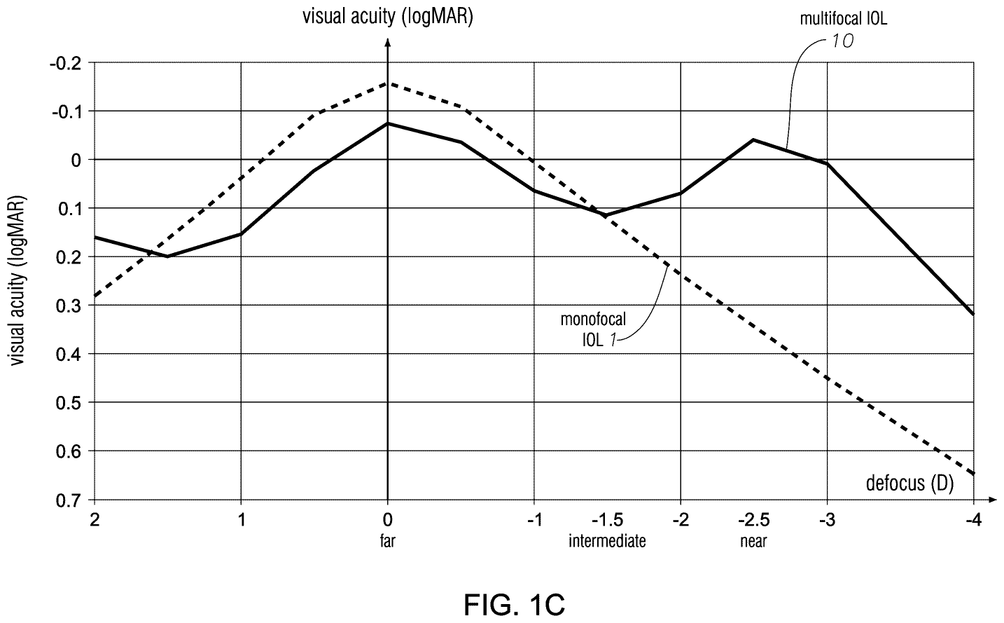

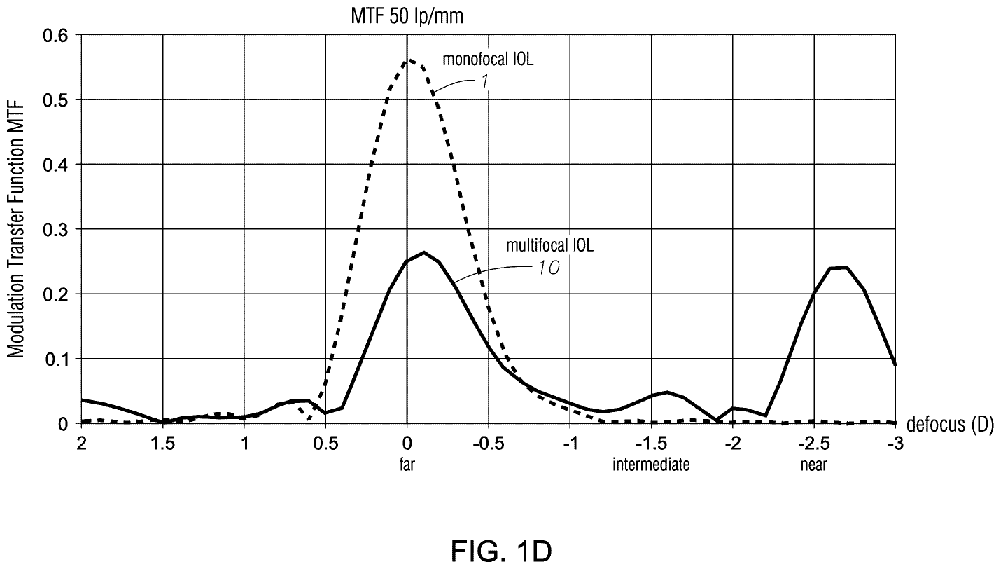

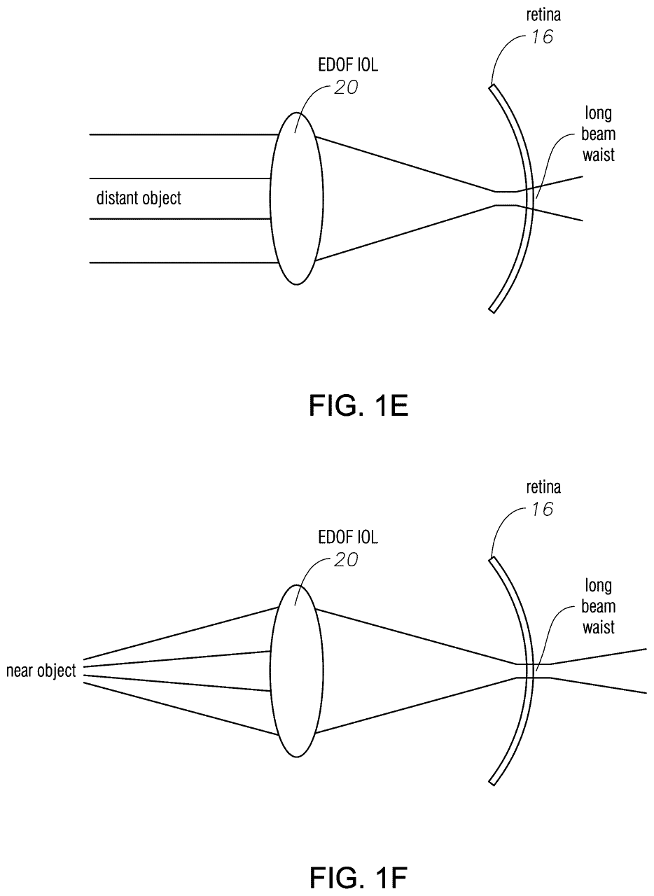

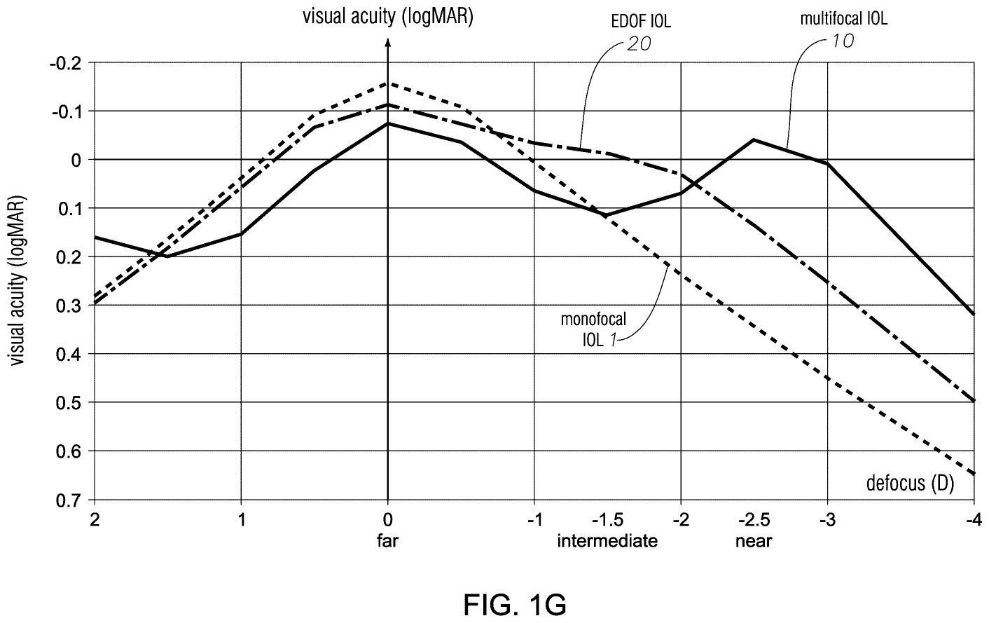

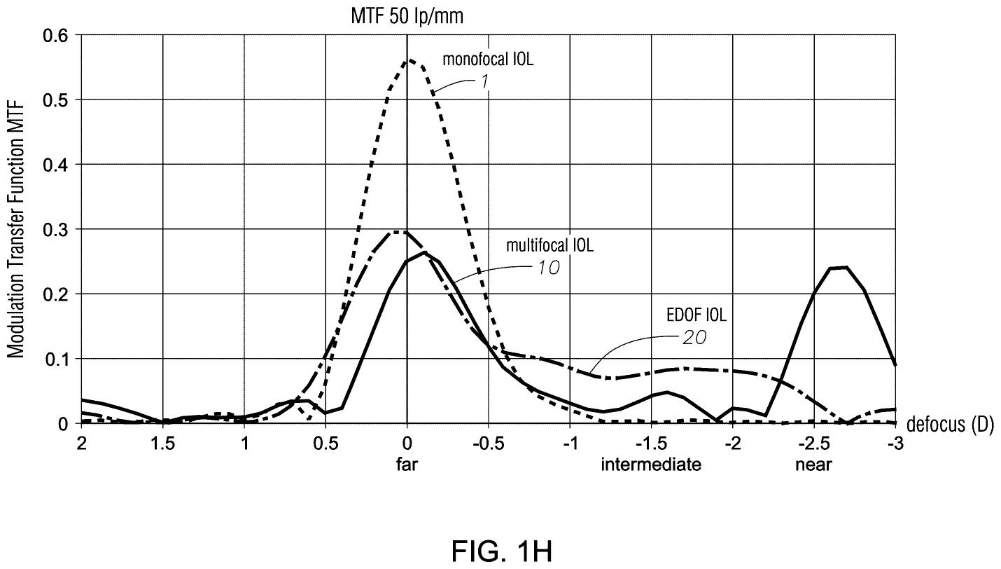

In what follows, new intraocular lens designs will be described that address the above challenges and deliver markedly improved optical performance for the benefit of patients. FIGS. 1A-B show the beam shape of existing multifocal intraocular lenses 10 for distant and near objects that have a center near add region 12 for near vision and the remaining peripheral region 14 for distance vision. Both of these regions have narrow beam waists and thus well-defined optical power, and therefore each image near and far objects, respectively, onto the retina 16 with high quality.

FIGS. 1C-D show two ways of characterizing the quality of the imaging of IOLs 10. FIG. 1C shows the visual acuity in terms of a “log MAR” plot that stands for a “logarithm of Minimum Angle Resolution”. The angle is in units of arcminutes and the logarithm is base 10. These plots are typically determined by giving a vision test to a patient in the “lane” of the office of the optometrist. Often a capital E is shown, and the patient is asked to report the orientation of the E. Determining this orientation requires visually resolving the three repeating lines of the E, and thus indicates a threshold of the patient's ability to resolve angular structures. Alternatively, regular letter charts are also widely used. This involves presenting a patient with letters of varying size at a fixed distance and determining the smallest letter sizes that the patient can resolve. log MAR plots can be also estimated without patient input, purely via optical modeling and ray tracing. In such a modeling approach, the perceptual elements of visual performance can be represented with empirical functions.

Broadly, log MAR 0.0 represents the patient resolving 1 arc minute at the lane distance. Better visual acuity means that the patient can resolve features of less than 1 arc-minute. The logarithm of numbers less than 1 is negative, and therefore increasingly negative log MAR values represent increasingly better visual acuity (VA). Therefore, increasingly negative log MAR values are conventionally plotted in the +y direction of an x-y plot. On the horizontal axis the negative inverse of the distance to the object (the lane distance) is plotted, in units of diopters D, or 1/meters, and referred to as the defocus d, or simply defocus. Put in a different way, the horizontal axis shows the power of a lens which replicates the focus condition. For example, the rays coming from an object that is 1 m away can be replicated by placing an object at infinity (0 D) and then placing a −1 m focal length lens in front of the eye. The power of this replicating lens is 1/(−1 m)=−1 D and so the focus condition is represented as d=−1 D on the horizontal defocus axis. The defocus d is increasingly negative along the defocus axis in the +x direction. Finally, it is also an industry standard to characterize the visual acuity at three distances: at far distance, translating to a defocus of 0 D; at intermediate distance of 0.66 m, i.e. at a defocus of 1.5 D, and at near distance of 0.40 m, i.e. at a defocus of 2.5 D. It is customary to refer to these three distances simply as distance (far), inter (intermediate), and near, dropping the “distance” altogether.

FIG. 1C shows that the log MAR plots of multifocal IOLs 10 exhibit two or more distinct peaks; one at distance/far at a defocus of 0 D, and one or more at near distances such as at a defocus of −2.5 D. The log MAR peak at near enables the multifocal IOLs 10 to mitigate the visual acuity of presbyopic patients who lost accommodation at near. However, this high visual acuity at near and distance/far comes at the price of a poorer visual acuity at intermediate distances, at a defocus of d=−1.5 D.

FIG. 1D shows an alternative way of characterizing visual acuity, through the Modulation Transfer Function, or MTF. The determination of the MTF does not involve patient feedback, and can be done by optical modeling of the IOL and eye's optical system, specifically the cornea. The MTF involves calculating a modulation amplitude of a periodic image in the focal plane of the IOL, when the IOL is imaging an object with a full, or 100% modulation. A typical example of a periodic object is a set of repeating stripes or lines. As such, the MTF is not a threshold criterion, unlike the log MAR.

The MTF has multiple variables. One of them is v, the spatial frequency of the periodic object, often expressed in terms of “line-pair/millimeter, or “lp/mm”: how many line pairs are within a millimeter of the imaged object. The MTF(v) can be plotted as a function of the spatial frequency v in units of lp/mm. These MTF(v) curves decrease as a function of decreasing object distance (for distance-corrected patients), i.e. increasingly negative defocus, because the image is moving away from the focal plane. FIG. 1D shows that the MTF can be also plotted at a fixed spatial frequency v at the focal plane, such as at 25, 50, or 100 lp/mm, as a function of the defocus d. FIG. 1D shows the MTF(d) at v=50 lp/mm. As before for the log MAR plots, increasingly negative defocus values correspond to increasingly close objects. The MTF(d) shows that a monofocal IOL 1 provides sharp visual acuity at d=0 D, when the image is in the focal plane, but this MTF(d) decreases as the object gets closer and thus the image moves away from the focal plane. On the other hand, the MTF of a multifocal IOL 10 has a second maximum when the object moves so close to the multifocal IOL 10 that its image by the center near add region 12 falls onto the plane corresponding to the distance focal plane of the peripheral region 14. This second maximum helps presbyopic patients to see near objects with good acuity even in the absence of accommodation by their own natural crystalline lens. Visibly, this benefit comes at a cost: delivering the good MTF at near reduces the MTF at distance. In FIG. 1D, the MTF(d=0) of the multifocal IOL 10 is reduced from the typical 0.55 of a monofocal IOL 1 to about 0.25, an about 50% reduction. Importantly, this reduction of the visual acuity cannot be recovered by wearing glasses. Wearing glasses only shifts the MTF laterally along the defocus axis. Therefore, wearing glasses would only help if there were another, higher peak of the MTF at some other defocus. But FIG. 1D shows that the MTF of the multifocal IOL 10 has no peak higher than the one at d=0 defocus. This makes the loss of visual acuity at distance irrecoverable. In addition, multifocal IOLs 10 have as low visual acuity at intermediate distances as monofocal IOLs 1—this is an additional problem for presbyopic patients. For completeness it is mentioned that multifocal IOLs 10 have different types, sometimes called diffractive, refractive, or zonal types, and the MTF and log MAR curves of different types can differ and exhibit variations.

FIGS. 1E-F show an IOL design that offers improved visual acuity at intermediate distances. FIGS. 1E-F show the beam shape for these Extended Depth of Focus, or EDOF IOLs 20 for distant and near objects. In these EDOF IOLs 20 the optical power progressively changes from a higher value at the center to a lower value in the peripheral region, in effect smoothing out the sharp change-of-power feature of the multifocal IOLs 10 at the outer radius of the center near add region 12. This smoothing out lengthens the beam waist of these EDOF IOLs 20.

FIGS. 1G-H show that this elongated beam waist of the EDOF IOLs 20 improves the visual acuity at intermediate distances, as demonstrated by the log MAR(d) and the MTF(d) being higher at d=−1.5D than the corresponding values of the multifocal IOLs 10. In addition, the visual acuity is essentially unchanged at far/distance at d=0, still well below that of monofocal IOLs 1. Visibly, though, the EDOF IOLs 20 deliver this substantial improvement at intermediate distances by substantially lowering the visual acuity at near. Nevertheless, the gain at intermediate distances is a primary reason why recently EDOF IOLs 20 attracted considerable interest and an increasing market share of late. At the same time, the decrease of the VA at near and the still-low acuity at far/distance is an ongoing source of persistent patient dissatisfaction.

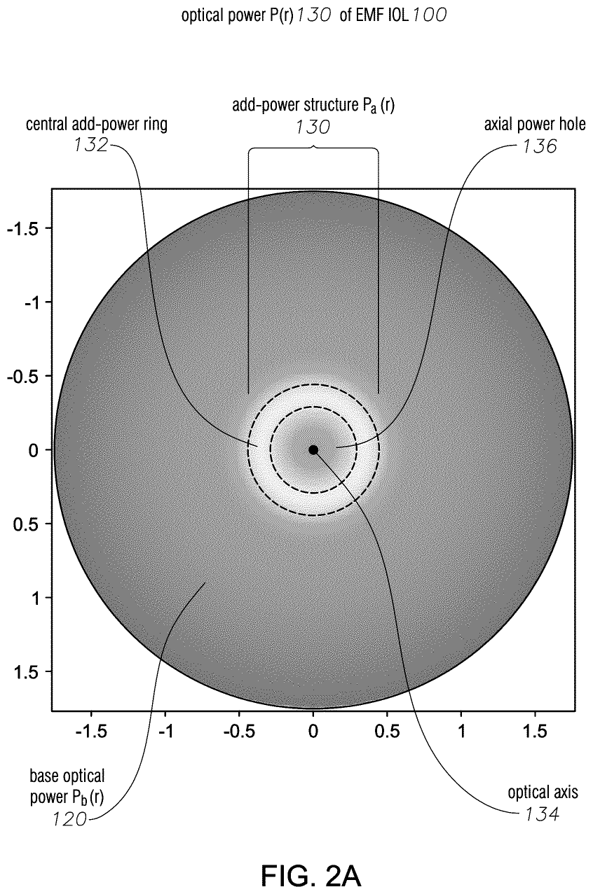

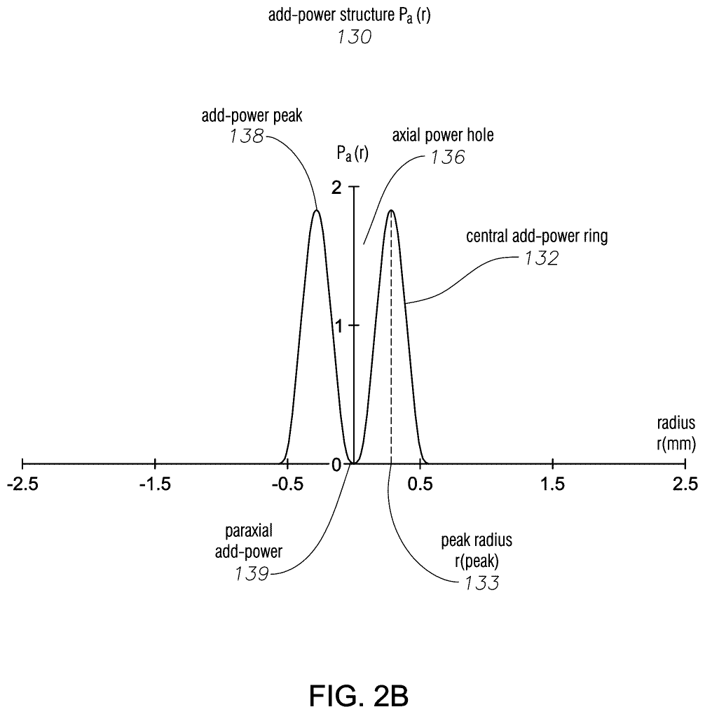

FIGS. 2A-B show embodiments of an Enhanced MonoFocal (EMF) IOL 100 that promises improvements over the above limitations. This EMF IOL 100 has a (total) optical power P(r) 110, which can be a sum of a base optical power P_{b}(r) 120 for distance vision consistent with a monofocal lens; and an additive add-power structure Pₐ(r) 130 for near vision, including a central add-power ring 132 around an optical axis 134 of the EMF IOL 100 that encloses an axial power hole 136, centered on the optical axis 134 of the EMF IOL 100. In formula:

$$P ( r ) = {P}_{a} ( r ) + {P}_{b} ( r ) \qquad ( 1 )$$

The (total) optical power P(r) 110 can be related to another quantity of importance, often called optical path difference OPD(r), optical path length, or wavefront W(r), which characterizes the length of the path the light rays traverse, multiplied by the index of refraction of the medium they traversed. The two are closely related by the relation:

$$P ( r ) = - \frac{1}{r} \frac{\partial W ( r )}{\partial r} \qquad ( 2 )$$

and the notation of (total) wavefront W(r) 111, base power wavefront W_{b}(r) 121 and add-power wavefront Wₐ(r) 131 will be adopted.

In different EMF IOLs 100, the base optical power P_{b}(r) can be consistent with a monofocal lens in various ways. In some EMF IOLs 100, the base optical power P_{b}(r) 120 can be characterized by a radius-independent monofocal optical power: P_{b}(r)=const. In other embodiments, the base optical power P_{b}(r) 120 can be characterized by a radius-dependent optical power with a corrective aberration that compensates a corneal aberration, at least partially. The cornea is known to introduce an aberration into the wavefront. This spherical aberration is positive, and its numerical value depends on the size/radius of the aperture over which it is being measured. In the corneal plane, at 3 mm radius, this corneal aberration is about 0.27-0.30 micron. Some EMF IOLs 100 can be designed to compensate this aberration partially, or fully, by incorporating fourth/sixth order Zernike polynomial terms into the base power wavefront W_{b}(r) 121 that induce a compensating negative spherical aberration:

$${W}_{b} ( r ) = {Σ}_{n = 2}^{4 or 6} {a}_{n} {Z}_{n} ( r ) \qquad ( 3 )$$

Some EMF IOLs 100 may even overcompensate the corneal aberrations, so that the cornea plus EMF IOL system exhibits a negative spherical aberration. The addition of negative spherical aberrative terms to the base power wavefront W_{b}(r) 121 induces a modest center-to-edge power variation of the base power P_{b}(r) 120, whose magnitude can be a few tenths of a diopter, such as 0.2 D, or 0.5 D, possibly rising to 1 D for EMF IOLs 100 that fully compensate or overcompensate the corneal aberrations. Since this power variation is only induced by the aberration compensation, these EMF IOLs 100 can still be referenced as being consistent with being a monofocal lens.

FIG. 2A is a 2D shading plot of the total P(r) 110, and FIG. 2B is a radial cross section of the add-power structure Pₐ(r) 130 of an embodiment of the EMF IOL 100—which will be often referenced simply as add-power Pₐ(r) 130, for brevity. Here the add-power Pₐ(r) 130 plot shows that the central add-power ring 132 has an add-power peak 138 at some peak radius r(peak) 133, and the axial power hole 136 at the optical axis 134 at r=0 has a paraxial add-power 139. The paraxial add-power 139 can be zero, or a small value, as discussed below. As shown, both the total optical power P(r) 110 and the add-power structure Pₐ(r) 130 have a minimum in the axial power hole 136 at the optical axis 134, and a maximum add-power peak 138 in the central add-power ring 132.

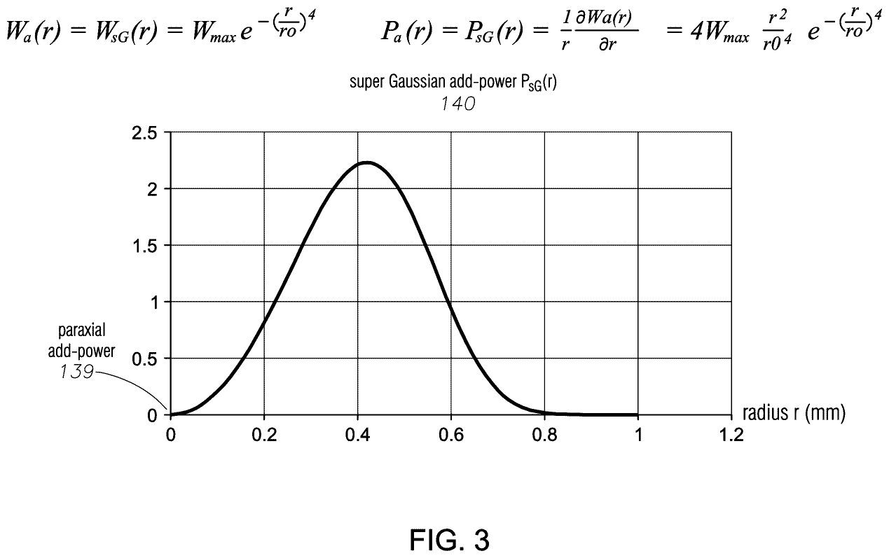

In some embodiments, the central add-power ring 132 of the add-power Pₐ(r) 130 has a maximum at the add-power peak 138 in a range of 1.0-4.0 diopters D; in other embodiments, in a range of 2.0-3.0 D. FIG. 3 shows an embodiment where the add-power peak 138 of the add-power Pₐ(r) 130 is about 2.2 D. Higher add-power peaks 138 in the range of 2.5-4.0 D can provide a visual acuity better at near distances but worse at intermediate and far distances. Lower add-power peaks 138 in the range of 1.0-2.5 D can provide a visual acuity less satisfying at near distances but better at intermediate and far distances. The competing design choices can be weighed and optimized by the doctor's judgement and the patient's preferences.

FIG. 3 shows an embodiment, where the add-power structure Pₐ(r) 130 is a super Gaussian add-power P_{sG}(r) 140, induced by an optical path difference Wₐ(r) 131 that is a super Gaussian wavefront W_{sG}(r) 141, the super Gaussian having a lens radius r raised to a power k greater than two in the exponential:

$${W}_{a} ( r ) = {W}_{s G} ( r ) = {W}_{max} {e}^{- {( \frac{r}{r o} )}^{k}} \qquad ( 4 )$$

FIG. 3 shows an embodiment with k=4. The corresponding super Gaussian add-power structure P_{sG}(r) 140 is then given by:

$${P}_{a} ( r ) = - \frac{1}{r} \frac{\partial {W}_{a} ( r )}{\partial r} = {P}_{s G} ( r ) = 4 {W}_{max} \frac{{r}^{2}}{r {0}^{4}} {e}^{- {( \frac{r}{r 0} )}^{4}} \qquad ( 5 )$$

Visibly, in such “super Gaussian EMF IOLs” 100 with k>2, Pₐ(r) 130 has a pre-exponential power law term with exponent (k−2): ˜r^{(k-2)} that forces Pₐ(r=0), the paraxial add-power 139, to be zero at r=0, thereby creating a complete axial power hole 136. A very extensive optimization was performed regarding the weighting factors of the various constraints. It was found that these super Gaussian embodiments of the EMF IOLs 100 caused the smallest distortion of the wavefront relative to monofocal IOLs 1, and thus kept their MTF(d=0) and log MAR(d=0) at the distance defocus d=0 closest to the monofocal values. This is a reason why the EMF IOLs 100 can be called enhanced monofocal IOLs.

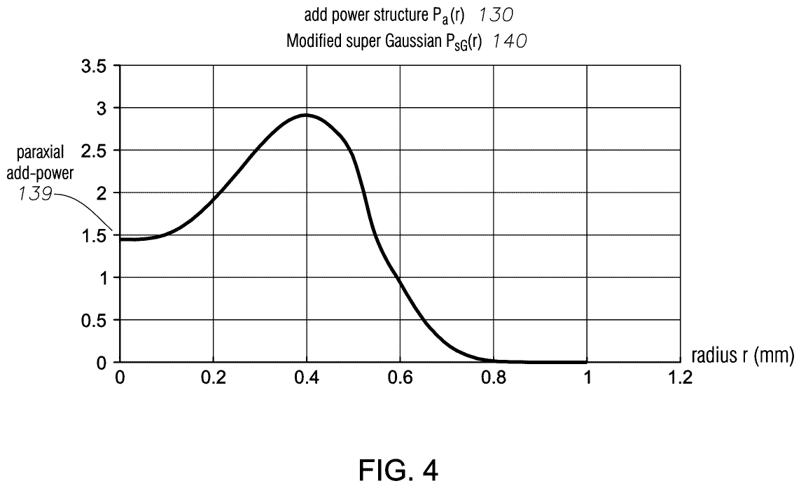

FIG. 4 shows that in other embodiments of the EMF IOLs 100, the axial power hole 136 may in fact add some paraxial power 139 to the paraxial base power P_{b}(0). Broadly, the axial power hole 136 can add a nonzero paraxial power 139 that is less than the (maximum) peak power 138 of the central add-power ring 132. Hence the name axial power hole 136: the paraxial power 139 of these EMF IOLs 100 is still less than their peak power 138, and therefore, when the total power P(r) 110 is plotted as a function of the radius r, P(r) 110 is not a monotonously decreasing function. Rather, the total P(r) 110 exhibits a power minimum at r=0, and then a power maximum at the peak radius r-r(peak)>0 133. For r>r(peak) Pₐ(r) 130 is a decreasing function, rapidly decaying towards zero, so that P(r) 110 will become equal to P_{b}(r) 120. In some embodiments, the peak radius r(peak) 133 does not exceed 0.5 mm; in others, 0.75 mm. Therefore, EMF IOLs 100 are clearly distinct from some existing EDOF IOLs, whose power is a monotonously decreasing function of the increasing radius r.

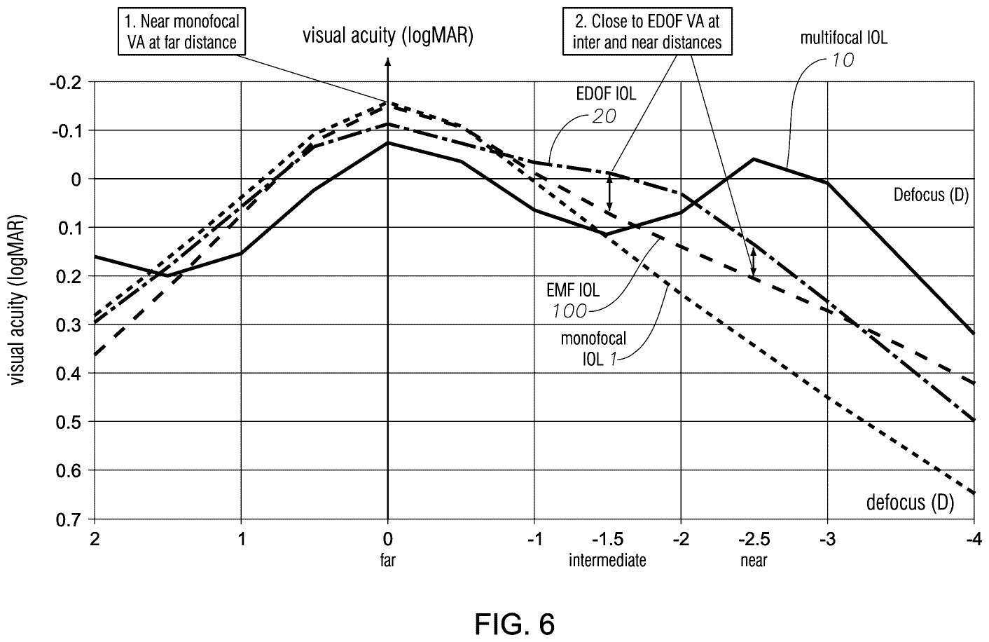

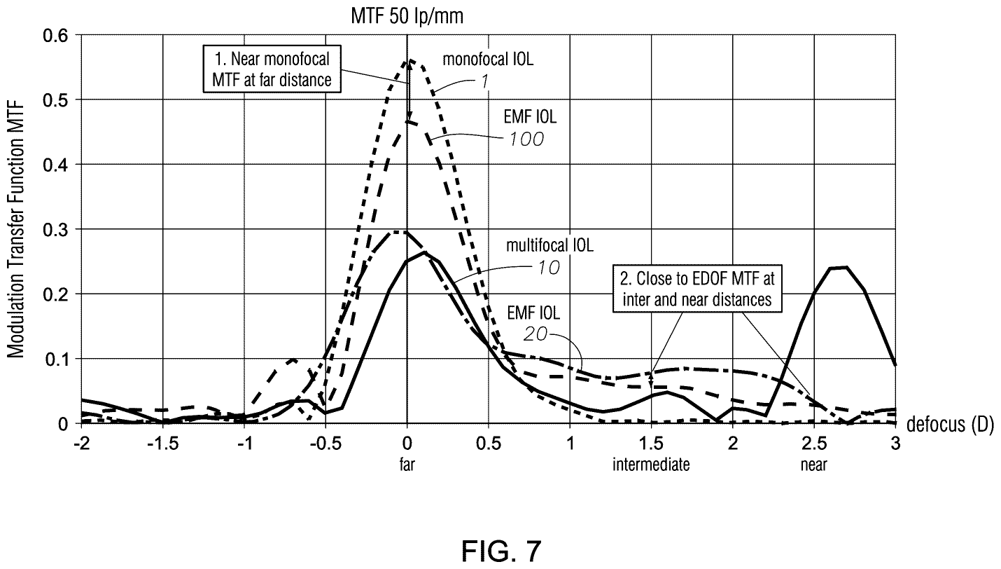

In some embodiments, the axial power hole 136 can add a nonzero paraxial add-power Pₐ(0) 139 less than 1 diopter D to the paraxial base power P_{b}(0). In other embodiments, the axial power hole 136 can add a paraxial add-power Pₐ(0) 139 less than 0.5 D to the paraxial base power P_{b}(0). The EMF IOL 100 embodiment in FIG. 4 shows an added paraxial add-power 139 Pₐ(0)=1.5D. This degree of design freedom of independently controlling the paraxial add-power 139 can be a reason to call these EMF IOLs 100 having “paraxial power control”. Advantages of these EMF IOLs 100 will be further analyzed in relation to FIGS. 6-7. As mentioned before, pure super Gaussian wavefronts W_{sG}(r) have zero paraxial add-power 139. Therefore, wavefronts that have a finite paraxial add-power 139 are super Gaussians that are modified with a corrective term that can generate a non-zero paraxial add-power 139.

It is notable that the techniques and devices used to characterize IOLs, such as wavefront aberrometers, have a finite spatial resolution, often as much as several tenths of a millimeter. This resolution can be comparable to the small spatial features of the EMF IOLs 100, such as to r(peak) 133, the radius of peak power, which can be as little as r(peak)=0.4 mm-0.5 mm in some embodiments. Therefore, the following can occur for an EMF IOL 100 that has zero paraxial add-power 139 Pₐ(0)=0 D, and thus a complete axial power hole 136, and a substantial add-power peak 138 Pₐ(r(peak)), say 2 D, at r(peak) 133. When such an EMF IOL 100 is characterized by a limited resolution wavefront aberrometer, the limited resolution of the measurement can seemingly “smear out” the axial power hole 136 and the add-power peak 138. In addition, the 1/r prefactor in the definition of the optical power amplifies the measurement errors as r is decreasing towards zero, which can have an additional smearing effect. Such a measurement can therefore report an incomplete axial power hole 136 with Pₐ(0)>0 and a reduced add-power peak Pₐ(r(peak))<2D, instead of the true values of Pₐ(0)=0D, and Pₐ(r(peak))=2D. Accordingly, in such limited resolution measurements, an EMF IOL 100 can appear to have a finite paraxial add-power 139 Pₐ(0)>0, even though a high resolution aberrometer would measure the true paraxial add-power 139 Pₐ(0) to be zero.

Given the just-demonstrated importance and usefulness of characterizing the EMF IOLs 100 in terms of their optical path distance/length, or wavefront W(r), we can characterize the EMF IOLs 100 directly as having an optical path difference W(r) 111 characterized by a base wavefront W_{b}(r) 121 for distance vision, and an additive add-power structure wavefront Wₐ(r) 131 for near vision, following an approximate super Gaussian W_{sG}(r) 141. The base wavefront W_{b}(r) 121 corresponds to the base optical power P_{b}(r) 120, and the additive add-power structure wavefront Wₐ(r) 131 corresponds to the add-power structure Pₐ(r) 130 of the optical power P(r) 110.

FIG. 3 shows that the super Gaussian wavefront W_{sG}(r) 141 can have a Gaussian exponential form with a radial coordinate raised to a power greater than two in the exponential. The terms radius and radial coordinate will be used interchangeably.

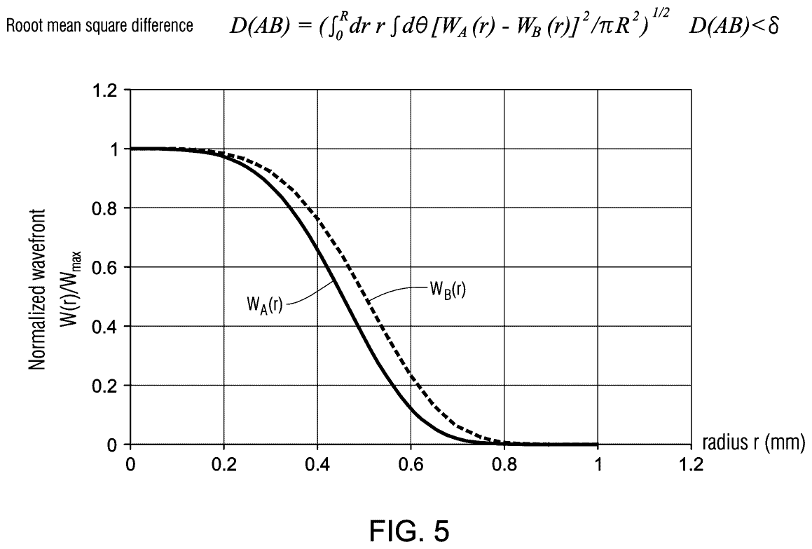

A paradigmatic factor that drives the superior extended depth of focus of these EMF IOLs 100 is the similarity of their add-power structure wavefronts Wₐ(r) 131 to a suitably parametrized super Gaussian wavefront W_{sG}(r) 141. Therefore, in the above description the embodiments were not defined only as those that strictly follow the specific super-Gaussian forms. Rather, that their optical powers Pₐ(r) 130 and wavefronts Wₐ(r) 131 approximately follow these forms. Such EMF IOLs 100 with wavefronts that approximate that of a super Gaussian wavefront W_{sG}(r) 141 also exhibit superior visual acuity and depth of focus properties. This approximate property can be quantified in different ways, including the widely used way to quantify the difference, or the closeness, between two wavefronts W_{A}(r) and W_{B}(r): via their root mean square difference D(AB). As shown in FIG. 5, D(AB) can be defined as:

$$D ( AB ) = {( {\int }_{0}^{R} d r r \int d {θ [ {W}_{A} ( r ) - {W}_{B} ( r ) ]}^{2} / π {R}^{2} )}^{1 / 2} \qquad ( 6 )$$

$$D ( AB ) = {( {\int }_{0}^{R} d r r \int d {θ [ {W}_{A} ( r ) - {W}_{B} ( r ) ]}^{2} / π {R}^{2} )}^{1 / 2}$$

Since the unit of W(r) is length, so is the units of D(AB). Given its relevance in calculating diffraction and wave propagation in general, it is customary to describe the wavefront W(r), and this optical path difference D(AB), as a fraction of the typical wavelength of the problem λ. For human vision, the relevant wavelength range is the visible spectrum of the sunlight, whose maximum is around λ=550 nm. Therefore, it is customary to talk about D(AB) being, for example, “a half wave”, which can mean D(AB)=0.5*550 nm, or any suitably chosen “design wavelength”. Accordingly, a dimensionless characterization of the mean square difference between wavefronts A and B can be the ratio of D(AB) to the typical wavelength λ: D(AB)/λ.

FIG. 5 shows that, with this preparation, embodiments of the EMF IOL 100 can have an add-power structure wavefront Wₐ(r) 131 that is well-approximated by a suitably parametrized super Gaussian wavefront W_{sG}(r) 141 such that D(a,sG)/λ<δ, where δ can be 0.05, 0.1, or 0.2 in some embodiments. Here, and below, “suitably parametrized” means that it is possible to construct a best-approximating super Gaussian wavefront W_{sG}(r) 141 with such parameters that the root mean square difference D(a,sG) between the best approximating super Gaussian wavefront and the add-power structure wavefront Wₐ(r) is less than δ in units of a wavelength of λ=550 nm: D(a,sG)/λ<β. Here, δ is 0.05, 0.1, or 0.2. In informal terms, this means that it is possible to find parameters for a super Gaussian wavefront W_{sG}(r) 141 that it will be so close to the add-power structure wavefront Wₐ(r) 131 of the EMF IOL 100 that D(a,sG)/λ<δ, with δ is 0.05, 0.1, or 0.2.

In some embodiments of the EMF IOLs 100, the super Gaussian W_{sG}(r) 141 of the add-power structure wavefront Wₐ(r) 131 is modified to generate a non-zero paraxial add-power P(0) 139 in a range of 0 D-1 D.

Our simulations found that very good visual acuity was achievable among EMF IOLs 100, whose power k in the exponent of the super Gaussian was in a range of 3.5-4.5.

FIGS. 6-7 summarize some of notable advantages of an EMF IOL 100 in terms of the previously introduced measures of Visual Acuity: the log MAR and the Modulation Transfer Function MTF graphs.

(1) As a result of a very extensive optimization of the constraint weight factors, the visual acuity of the EMF IOLs 100 at distance/far is remarkably close to monofocal IOLs 1, and notably enhanced relative to EDOF IOLs 20. For example, the MTF_{EMF}(d=0) of the EMF IOL 100 of FIG. 7 is only 15% lower than the MTFₘₒₙₒ(d=0) of a corresponding monofocal IOL 1 with the same base optical power alone. In other embodiments, MTF_{EMF}(d=0) can be within 30% of the MTFₘₒₙₒ(d=0). This is a much better optical performance than that of the EDOF IOLs 20, whose MTF_{EDOF}(d=0) can be reduced by 45% relative to the distance mono MTFₘₒₙₒ(d=0).

(2) Even though the EMF IOLs 100 deliver an MTF at far defocus that is markedly superior to that of EDOF IOLs 20, they are still able to achieve a visual acuity at intermediate and near that is comparable to that of EDOF IOLs 20 because of their smooth super Gaussian design of the add-power structure Pₐ(r) 130, and of our extensive optimization. FIG. 6 shows that the log MAR of the EMF IOLs 100 is only about 0.05 lower at intermediate and near defocus than that of the EDOF IOLs 20. FIG. 7 shows that the MTF of the EMF IOLs 100 is essentially indistinguishable from the MTF of the EDOF IOLs 20 at intermediate and near distances.

(3) When compared to monofocal IOLs 1 instead at intermediate and near, the add-power structure Pₐ(r) 130 extends the depth of focus of the EMF IOL 100 relative to the depth of focus of monofocal IOLs 1 with the corresponding base power P_{b}(r) 120 alone. One way to quantify this extension of the depth of focus is that, the negative log MAR of the EMF IOLs 100 of FIG. 6 exceeds 0.2 over a longer range of diopters for the EMF IOL 100 with the base optical power P_{b}(r) 120 plus the add-power structure Pₐ(r) 130, than for the monofocal IOL 1 with the corresponding base optical power P_{b}(r) 120 alone. In FIG. 6, the negative log MAR exceeds 0.2 in the 0D-(−2.5D) region for the EMF IOL 100, while it exceed 0.2 only in the 0D-(−1.8D) range for the monofocal IOL with the base power P_{b}(r) 120 alone. (Here we used the term “negative log MAR exceeds” to refer to log MAR values that appear in the +y direction, “above”, relative to 0.2.)

(4) All these benefits are achieved while minimizing the risk of generating halos. Halos in existing EDOF IOLs 20 can be generated by diffractive structures and sharp edges, which the EMF IOLs 100 do not have. In addition, myopia-related halos can also be eliminated by the subsequently discussed light adjustment procedure.

These advantages are due to several design differences between existing EDOF IOLs 20 and the EMF IOLs 100, as discussed next.

(1) Wide classes of EDOF IOLs 20 have a central bump such that their optical power P(r) 110 has a maximum at r=0, and monotonously decreases with increasing radial coordinate r. In contrast, the add-power structure Pₐ(r) 130 of the EMF IOLs 100 has an axial power hole 136, and so their add-power structure Pₐ(r) 130, and therefore the total optical power P(r) 110 has a minimum at r=0, and increases for small r, not decreases. Therefore, the overall variation of the total optical power P(r) 110 is a non-monotonous function of the radial coordinate r of the EMF IOL 100 such that the optical power P(r) 110 has a minimum in the axial power hole 136, and a maximum in the central add-power ring 132. Typically, the optical power P(r) 110 has its minimum at the center at r=0, its maximum at the peak radius r(peak) 133, and decreases monotonously for r>r(peak).

(2) Other existing EDOF IOLs 20 have a more complex central add structure 12, with phase shift structures whose optical power decreases, then increases, and then decreases again with the radial coordinate r. These structures have an optical power that has a minimum at a finite radial coordinate r. Their add power Pₐ(r) 130 is typically negative around this minimum. Some have a toroidal, or ring shaped ridge on the surface of the lens itself, i.e. in W(r), not in the optical power P(r), as in the EMF IOLs 100. In contrast, the EMF IOLs 100 do not have a power minimum at a finite radial coordinate r. Their W(r) does not have a toroidal shape either: W(r) has a maximum at r=0, and decreases monotonically with increasing radial coordinate r. Accordingly, the add-power structure Pₐ(r) 130 of the EMF IOLs 100s is non-negative at all radial coordinates: it has no region of negative power.

(3) Many of these phase shift structures share a linear variation of their wavefront, some of them in a segmented, multi-region pattern. In contrast, EMF IOLs 100 do not have linear segments in their wavefronts. Using the above characterization tool D(a,b), a root mean square difference D(a,lin) between the add-power structure wavefront Wₐ(r) 131 and a best fitting linear wavefront Wₗᵢₙ(r) with a central flat top and one decreasing linear segment exceeds Δ in units of a design wavelength of λ=550 nm: D(a,lin)/λ>Δ, wherein Δ=0.1, 0.2 or 0.5.

(4) The radius of this central add phase shift structures of these existing EDOF IOLs 20 typically exceeds 1 mm. In contrast, EMF IOLs 100 have a smaller characteristic, peak radius r(peak) 133. Our simulations have shown that, while central near add structures 12 of such large radii may deliver good near vision, they compromise distance vision to an uncomfortable degree. Motivated by these simulations, the add-power structures Pₐ(r) 130 of the EMF IOLs 100 are implemented with an unusually small characteristic radius where their add-power peak 138 is located at a peak radius 133 of r(peak)=0.5 mm or less. Some embodiments found benefits of r(peak) 133 being as big as 0.75 mm, but all these values are still markedly below 1 mm.

(5) The total wavefront W(r)=Wₐ(r)+W_{b}(r) of the EMF IOLs 100 are substantially different from most existing EDOF IOLs 20. Indeed, the wavefronts of most EDOF IOLs 20 are characterized by Zernike polynomials Zₙ(r), and their wavefront W(r) is defined as a sum of a few leading Zernike polynomials:

$$W ( r ) = {Σ}_{n = 2}^{N} {a}_{n} {Z}_{n} ( r ) \qquad ( 7 )$$

where N is typically 4 or 6, and does not exceed 10, or 12 at most. In contrast, the EMF IOLs 100 have add-power structure wavefronts Wₐ(r) 131 that are described in terms of super Gaussian wavefronts W_{sG}(r) 141. When one attempts to describe the add-power structure wavefront Wₐ(r) 131 of the EMF IOL 100 in terms of Zernike polynomials, one often needs to go out to N=20 or higher. This means that if one attempts to describe the add-power structure wavefront Wₐ(r) 131 with W_{ZN}(r), a sum of Zernike polynomials truncated at N=8, 10 or 12, then the remaining differential, unaccounted for by Zernike polynomials is still a substantial fraction of the add-power structure wavefront Wₐ(r) 131 over the area of the EMF IOL 100. In some EMF IOLs 100, the above defined measure of wavefront differences, the root mean square difference D(a,ZN) between the Wₐ(r) 131 of the EMF IOL 100 and that of the best approximating sum of Zernike polynomials truncated at N, W_{ZN}(r), is still greater than Δ, in units of the wavelength λ: D(a,ZN)/λ>Δ, where Δ is larger than 0.1, 0.2, or 0.5, for λ=550 nm, and for N=8, 10, or 12. Here N is the first index of the two index notation of the Zernike polynomials. If the single index notation is used for the Zernike polynomials, then the N in the D(a,ZN) can be as high as 15 or even 20.

This incompatibility has a mathematical reason. The Zernike polynomials Zₙ(r) with increasing indices n have the radius r raised to higher and higher exponents n. As such, they produce contributions to Wₐ(r) 131 that increase more and more with r. In contrast, the EMF IOLs 100 have an add-power structure wavefront Wₐ(r) 131 that exponentially decreases at large r. It takes a large number of Zernike polynomials (that increase with r) to delicately counterbalance each other and produce the exponentially decreasing Wₐ(r) 131 of the EMF IOLs 100.

(6) Another advantage of the EMF IOLs 100s is that large classes of EDOF IOLs 20 are designed by optimizing their central add-power structure and their peripheral base power separately and independently from each other. When finally their add-power structure and the peripheral base power structure are connected at some r(con) connecting/matching radius, there is often a mismatch, a step in the power, or a jump or kink in the wavefront because the design of the two regions was carried out separately. In such lenses, a crucial design step is to smooth over this step, kink, or jump. This matching/smoothing step often degrades their overall visual acuity. In contrast, the base optical power P_{b}(r) 120 and the add-power structure Pₐ(r) 130 in the EMF IOLs 100 are simply additive. No matching or smoothing is required at any radius, because by design there was no kink, jump or step in these lenses between the central and the peripheral regions in the first place. Both the base wavefront W_{b}(r) 121 and the add-power structure wavefront Wₐ(r) 131 are defined over the entire range of radii, from r=0 to typically 3 mm.

(7) A wide class of IOLs achieve their design goals by using diffractive structures, gratings, grooves, or echelettes. In contrast, EMF IOLs 100 do not have diffractive structures, grooves or echelettes.

Finally, it is mentioned that there is an emerging new class of IOLs called Light Adjustable Lenses, or LALs. These were extensively described in a number of commonly owned patents, such as U.S. Pat. No. 6,450,642, entitled: “Lenses capable of post-fabrication power modification” to Jethmalani et al.; U.S. Pat. No. 10,874,505, entitled: “Using the light adjustable lens (LAL) to increase the depth of focus by inducing targeted amounts of asphericity”, to Sandstedt et al.; and U.S. Pat. No. 11,191,637, entitled: “Blended extended depth of focus light adjustable lens with laterally offset axes”, to Goldshleger et al., all incorporated in their entirety by reference. In such LALs, the add-power structure Pₐ(r) 130 of the EMF IOLs 100 can be either formed by a light adjustment procedure after implantation, or it can be pre-molded already during their manufacture, prior to implantation.

Such light adjustability brings additional benefits ibn the quest to provide optimized visual acuity. For example, some types of halo formation are related to the implanted IOL providing a myopic vision, especially in EDOF or multifocal IOLs. This can happen because the doctor did not select the correct IOL, or did not implant the IOL at the optimal or planned location. In either case, eliminating the unintended myopic effect after implantation can greatly reduce or eliminate this type of halo formation. Of the existing IOLs, only the light adjustable IOLs, or LALs allow the doctor to correct this unintended myopia after the surgery.

While this document contains many specifics, details and numerical ranges, these should not be construed as limitations of the scope of the invention and of the claims, but, rather, as descriptions of features specific to particular embodiments of the invention. Certain features that are described in this document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to another subcombination or a variation of a subcombinations.

1. An enhanced monofocal (EMF) intraocular lens (IOL), wherein:
an optical power of the intraocular lens is characterized by
a base optical power for distance vision, consistent with a monofocal lens; and
an additive add-power structure for near vision, including
a central add-power ring around an optical axis of the IOL; and
an axial power hole at the optical axis of the IOL.

2. The enhanced monofocal IOL of claim 1, wherein:
the base optical power is characterized by one of
a radius-independent monofocal optical power; and
a radius-dependent optical power with a corrective aberration that compensates a corneal aberration, at least partially.

3. The enhanced monofocal IOL of claim 1, wherein:
the optical power is a non-monotonous function of a radius of the IOL such that the optical power has
a minimum in the axial power hole; and
a maximum in the central add-power ring.

4. The enhanced monofocal IOL of claim 1, wherein:
the central add-power ring generates an add-power with a maximum in a range of 1.0-4.0 diopters.

5. The enhanced monofocal IOL of claim 1, wherein:
the central add-power ring generates an add-power with a maximum in a range of 2.0-3.0 diopters.

6. The enhanced monofocal IOL of claim 1, wherein:
the add-power structure is induced by an approximate super Gaussian optical path difference, the super Gaussian having a radial coordinate raised to a power greater than two in its exponential.

7. The enhanced monofocal IOL of claim 1, wherein:
the axial power hole generates a paraxial add-power less than 1 diopter.

8. The enhanced monofocal IOL of claim 1, wherein:
the axial power hole generates a paraxial add-power less than 0.5 diopter.

9. The enhanced monofocal IOL of claim 1, wherein:
a maximum of the add-power generated by the central add-power ring is at a radius that does not exceed 0.5 mm.

10. The enhanced monofocal IOL of claim 1, wherein:
the add-power structure extends a depth of focus of the EMF IOL relative to the depth of focus of an IOL with the same base optical power alone.

11. The enhanced monofocal IOL of claim 10, wherein:
a negative log MAR of the EMF IOL with the base optical power plus the add-power structure exceeds 0.2 over a longer range of defocus diopters than that of an IOL with the same base optical power alone.

12. The enhanced monofocal IOL of claim 1, wherein:
a modulation transfer function of the EMF IOL at zero defocus is within 30% of the modulation transfer function of a corresponding monofocal IOL with the same base optical power alone.

13. The enhanced monofocal IOL of claim 1, wherein:
a root mean square difference between a wavefront that induced the add-power structure and a best fitting sum of Zernike polynomials truncated at N=8 exceeds 0.2 in units of a wavelength of 550 nm.

14. The enhanced monofocal IOL of claim 1, wherein:
the add-power structure is non-negative for all radii.

15. The enhanced monofocal IOL of claim 1, wherein
the IOL is light adjustable; and
the add-power structure is premolded, or is formed by a light adjustment procedure.

16. An enhanced monofocal intraocular lens, wherein:
an optical path difference W(r) of the enhanced monofocal intraocular lens (EMF IOL) is characterized by
a base wavefront W_{b}(r) for distance vision; and
an additive add-power structure wavefront Wₐ(r) for near vision, approximately characterized by a super Gaussian W_{sG}(r), having a radial coordinate r raised to a power greater than two in its exponential.

17. The enhanced monofocal IOL of claim 16, wherein:
the add-power structure wavefront Wₐ(r) is approximately characterized by the super Gaussian W_{sG}(r) in the sense that a best-approximating super Gaussian can be found, for which a root mean square difference D(a,sG) between the best approximating super Gaussian wavefront and the add-power structure wavefront Wₐ(r) is less than δ=0.1, in units of a wavelength of λ=550 nm: D(a,sG)/λ<δ.

18. The enhanced monofocal IOL of claim 16, wherein:
the radius in the exponential of the super Gaussian is raised to a power between 3.5 and 4.5.

19. The enhanced monofocal IOL of claim 16, wherein:
the add-power structure wavefront induces an additive add-power structure for near vision, including
a central add-power ring around an optical axis of the IOL; and
an axial power hole at the optical axis of the IOL.

20. The enhanced monofocal IOL of claim 19, wherein:
the central add-power ring generates an add-power with a maximum in a range of 1.0-4.0 diopters.

21. The enhanced monofocal IOL of claim 16, wherein:
a root mean square difference between the add-power structure wavefront Wₐ(r) and a best fitting sum of Zernike polynomials truncated at N=8 exceeds Δ=0.1 in units of a wavelength of λ=550 nm: D(a,ZN)/λ>Δ=0.1.

22. The enhanced monofocal IOL of claim 16, wherein:
a root mean square difference between the add-power structure wavefront Wₐ(r) and a best fitting wavefront Wₗᵢₙ(r) with a central flat top and one decreasing linear segment exceeds Δ=0.1 in units of a wavelength of λ=550 nm: D(a,lin)/λ>Δ=0.1.

23. The enhanced monofocal IOL of claim 16, wherein:
the super Gaussian of the add-power structure wavefront Wₐ(r) is modified to generate a non-zero paraxial add-power in a range of 0 D-1 D.

24. The enhanced monofocal IOL of claim 16, wherein:
the entire add-power structure wavefront Wₐ(r) is a decreasing function of a radius r.

25. The enhanced monofocal IOL of claim 16, wherein
the IOL is light adjustable; and
the add-power structure wavefront Wₐ(r) is pre-molded or is formed by a light adjustment procedure.
